(19) United States
(12) Patent Application Publication
(10) Pub. No.: US 2025/0057629 A1
(43) Pub. Date: Feb. 20, 2025

(54) Slip-on Sleeve or Clip Attached to Medical Instruments to Provide Visualization

(71) Applicant: John A. Costin, Avon Lake, OH (US)

(72) Inventor: John A. Costin, Avon Lake, OH (US)

(21) Appl. No.: 18/802,756

(22) Filed: Aug. 13, 2024

Related U.S. Application Data
(60) Provisional application No. 63/520,472, filed on Aug. 18, 2023.
(60) Provisional application No. 63/520,492, filed on Aug. 18, 2023.
(60) Provisional application No. 63/520,481, filed on Aug. 18, 2023.
(60) Provisional application No. 63/520,458, filed on Aug. 18, 2023.
(60) Provisional application No. 63/580,033, filed on Sep. 1, 2023.
(60) Provisional application No. 63/602,759, filed on Nov. 27, 2023.

(51) Int. Cl. A61B 90/35; A61B 1/313; A61B 90/00

(52) U.S. Cl. CPC A61B 90/35; A61B 90/361; A61B 1/313

(57) ABSTRACT

A surgical device for carrying out surgery inside a human body has a surgical device that is inserted into a human body to carry out the surgery. The illumination and also a sensor such as a camera are set on an illumination holder that is clipped or slid over the surface of the device. This provides illumination and image sensor near the operative tip of the surgical device to image an area of the surgical device.

This application claims priority from all of:
Provisional application No. 63/520,472, filed Aug. 18, 2023,
63/602,759, filed Aug. 18, 2023,
63/520,492, filed Aug. 18, 2023
63/520,481, filed Aug. 18, 2023
63/520,458, filed Aug. 18, 2023
63/580,033, filed Sep. 1, 2023, and
63/602,759, filed Nov. 27, 2023,
the entire contents of each of which are herewith incorporated by reference.

### BACKGROUND

Surgeries and other medical procedures are continuing to transition to less invasive, smaller incisions with consequent less anesthesia, faster recovery and less pain. All of this is done in hopes of reducing healthcare costs, while lessening complications and associated morbidity and thus improving patient outcomes.

This transition is dependent upon and even mandates accurate visualization of the pathology and of the procedure. This visualization has been achieved via a visualization instrument separate from the surgical instrument.

A round cross section catheter is commonly used in different surgeries. In brain surgery, a barrel is inserted into the brain tissue. A visualization device and a suction catheter are inserted into the barrel. Adding more items into the catheter reduces the visualization.

As another example, an endoscope requires in general, a separate opening for visualization or a shared occupation of an opening with another instrument. This is often used in neurosurgery. An opening such as a craniotomy is preferred to be a single entry. The opening is typically viewed from the exterior with a microscope or loupes. Any instrument placed in the craniotomy opening lessens the observers' visualization distally into the brain from the microscope or loupes. With the objective of MIS (minimally, invasive surgery), the smaller sized opening, and less retraction of brain tissue are more desirable. However, additional items in this craniotomy opening reduce the surgeon's visualization.

### SUMMARY OF THE INVENTION

The inventor recognized, that when surgical instruments, such as a suction device, cautery or a grasper, are placed into the minimal space opened for the surgical procedure, the visualization is compromised. In some cases, the visualization is obscured to the potential detriment of the procedure.

In addition, often the retraction system gives access to the area of interest which can be a cavity or a cavern. Visualization of the extent and the architecture of this space is not possible with these approaches. Conventional visualization, where a sensor is extended separately down the retractor, also presents issues with space and instrument interference, and size of the craniotomy opening.

The present application describes, for the first time, placing a movable, adjustable micro sensor for visualization on a visualization holder, which can be a sheath or a clip directly on a shaft or arm of the instrument participating in the procedure or operation.

In an embodiment, a surgical device has its shaft or arm covered in a location with a moveable elastic sleeve or clip like device, with a high acuity visualization sensor embedded into this elastic sleeve or clip device so that the visualization sensor can be placed on the shaft of the instrument.

This inventive concept provides a clear breakthrough and allows for the critical visualization the surgeon needs to achieve much better outcomes,

In other embodiments, a sheath with a visualization sensor is attached to other surgical tools including forceps, graspers, cutters, and other tools.

The visualization is adjusted to get maximum views and minimal interference which are embodiments to the invention. The object of interest would be right at the target of the procedure. Exploration and/or mitigation would be easier and limited by the motility of the instrument itself, not the retractor, or the external visualization such as with loupes or with the microscope.

In embodiments, light to the target is either transmitted via the power cable conduit to the sensor or generated in or on the sensor itself allowing for only a small, thin power cable wire along the side of the instrument which are additional embodiments of the invention.

This inventive concept provides novel and improved patient outcomes in a variety of surgical fields including but not limited to neurosurgery by providing the necessary visualization at the target for a variety of different size surgical instruments and procedures.

This inventive concept of a sleeve, sheath, or clip allows placement of the visualization sensor(s) on and at the target of interest for more accurate placement and procedural outcome. Illumination of the sensor could be via a light path external to the device or on, in, or in proximity to the sensor(s).

### BRIEF DESCRIPTION OF THE DRAWINGS

The concepts in the Figures illustrate many embodiments of the invention.

FIG. 1 shows a suction device with visualization;

FIG. 2 Shows a shaft like instrument with a cutting tip and a hollow shaft to allow evacuation of material being treated;

FIG. 3 shows graspers with the visualization sensor and a sleeve;

FIG. 4 shows an alternative version of the graspers with the sleeves;

FIG. 5 shows a cannula, catheter, of any other means of delivering a device such as a balloon, a capsule, a stent, a mesh for occlusion, or a means of opening or widening a space or vessel;

FIGS. 6A and 6B show forceps with a visualization sleeve;

FIG. 6C shows a view of the forceps showing an opening 663 between facing sections; and

FIG. 7 shows a hand held cautery device with a visualization sleeve

### DETAILED DESCRIPTION

Surgical instruments vary in size and shape depending upon the procedure and surgeon. Many different surgical instruments are known, and all of them are intended to be encompassed within these embodiments described herein.

The embodiments show a visualization holder for the visualization hardware that is used according to the present application. As described herein, the visualization hardware can include illumination devices such as LEDs, and one or more cameras. The visualization holder is powered by a wire or wire assembly that is clipped on or attached to the surgical instrument as described in the embodiments.

In another embodiment, the visualization holder includes an internal battery, preferably a flat pack battery, and includes a wireless communicator for the information, thus making the assembly completely wireless and provide wireless visualization on the tool.

The visualization holder is formed of a covering that slides over the outer surface of an end portion of the surgical instrument. A miniaturized light source and camera are embedded or otherwise attached to the covering in an embodiment.

Providing the camera on the outside of the shaft of the surgical instrument provides significant advantages not present in the prior art. The sensor has a dramatic depth of field which allows aspiration or vacuuming without getting too close and obscuring the image. The visualization holder can be moved on the shaft, allowing the visualization to even further improve. Not only is the accuracy of the surgery greatly improved, but even smaller diameter tools can be used, and the visualization can allow the surgeon the place another instrument into the operative field with direct visualization provided by the visualization holder.

In a first embodiment, the covering is a sleeve that can be moved along the shaft of the surgical instrument by sliding the inside surface of the sleeve against the outside surface of the shaft of the surgical instrument. The sleeve is preferably formed of a flexible material that is snug against the outer surface of the shaft of the surgical instrument.

In a second embodiment, the covering is a clip that does not completely surround the shaft of the surgical instrument, but goes around most of the shaft, leaving an opening between the two edges of the clip, so that the covering can be clipped over the outer surface. The clip is also preferably formed of a flexible material snug against the outer surface of the shaft.

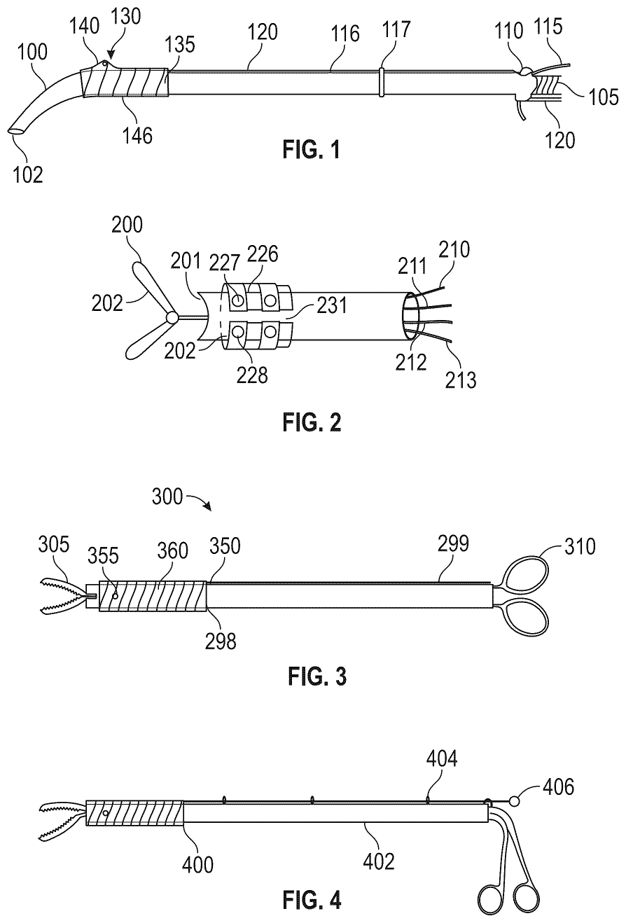

FIG. 1 shows an embodiment where the surgical instrument is a suction device. These suction devices are often associated with a bipolar or monopolar cautery function. Frequently a separate channel for irrigation is also present.

FIG. 1 shows an embodiment. The aspiration tube 100 has a tip 102 which is inserted into the opening used for the surgery. The tip 102 receives suction from a suction source 105 which is attached at the opposite end 110 of the tube, which also provides power from a power cable 115 and irrigation from irrigation source 120, to be used at the tip 102.

The power cable 115 can be a microsized power cable, for example 0.4 mm in diameter which is clipped on or attached with surgical glue as in 116 to the outside surface 120 of the tube 100. The power cable 116 runs to the visualization sensor assembly 130, which is formed of one or multiple LEDs 135, and to a visualization camera 140. LEDs 135 and visualization camera 140 are all microsized devices. In embodiments, the camera is 0.5-1 mm in diameter or width, preferably 0.9 mm. The wire can be 0.1-0.2 mm in diameter.

The LEDs 135 and the camera 140 are housed on the visualization holder, which in this embodiment is an elastic sleeve 146 that is flexible and movable. The inside surface of the elastic sleeve is slightly smaller than the outside surface 120 of the tube, so that the sleeve 146 fits snugly against the tube. The sleeve is formed of an elastic material, preferably an elastomeric material, so that a sleeve can fit around multiple different size devices.

The covering in this embodiment is an elastic sleeve 146 is placed over the outside surface 120 of the tube, and located into place, at the time of use where it is maintained in place by clips or glue or just by friction.

In one embodiment, the sleeve can be movable. In another embodiment, the covering may be held in place. When the covering is movable, it may be formed of an elastomeric material, which tightly fits to the outside of the tube 120.

The tube in FIG. 1 can be straight or curved as depicted.

In another embodiment, light is conducted down a small channel that is separate.

The covering could be made of a material, such as elastomeric material, that holds to the outside of the tube 120, to allow the sensor(s) to be moved up or down the shaft for optimal performance, such as to adjust the depth of field, the angle of illumination, or the angle of viewing. The suction/aspiration devices have varying diameter shafts and an elastic material that could accommodate these different diameters.

The camera 130 is placed close to the tip 102 for direct visualization during all functions. However, the position of the camera 130 can be adjusted on the tip to provide optimal clarity and depth of field. By being able to slide the sensor, blood, opaque fluids or plume from cautery or surgical manipulation could be minimized and allow less obstructed viewing.

FIG. 1 shows how the power cable can be a thin cable 116 run along the edge of the sensor, and can be clipped on with a clip 117. The cable can be a flat cable that can include multiple different lines, including power, common, and image lines.

The visualization can use a single chip visualization sensor with light illumination included. Alternatively, the visualization structure can use separate LEDs and camera chips.

FIG. 2 shows a shaft like instrument with a cutting tip 200 and a hollow shaft to allow evacuation of material being treated. The mechanical end 209 has an opening on the side of the cutting tip 200. or at the outer tip. The covering in this embodiment is the clip, which is an elastomeric device that does not extend all the way around, and has an opening 231 between its edges, allowing the covering to be clipped over the tube rather than slid down the tube as is required when the covering is a sleeve. The sleeve has sensors 225 and illumination LEDs 226, all powered by the wires running through the sleeve. The covering could be of other materials, such as metal.

The tube itself 205 is hollow, and receives power through the tube including sensor power 210, cutter power 211, aspiration and vacuum 212, and irrigation 213. The clip is clipped over the outside of the tube, with its embedded visualization sensor 225 an LED assembly 230.

In an embodiment, two opposing sensors 227, 228 are placed on a ring together to provide a wider field of view and possible stereopsis if desired.

FIGS. 3 and 4 shows embodiments where the surgical tool is graspers. The visualization holder in this embodiment of FIG. 3 is a visualization sleeve that is slid over and portion of the cannula that holds the surgical devices. The tool 300 has a grasper end 305 that is controlled by the control end 310 to provide conventional methods of articulation and articulation. The sleeve 350 is located over the tube to provide the light and imaging function. As in other embodiments, the sleeve 350 is formed of an elastic material, and includes a camera 355, and one or more LEDs 360 controlled by power that extends down the tube. In the embodiment of FIG. 3, the tube is a long tube 350. The embodiment of FIG. 4 shows a shorter tube 400.

The graspers can be forceps tips that are actuated through a shaft that is fixed on its outer wall. Forceps are used to grasp an item during a surgical operation. One particular forcep is often referred to as a bayonet forcep. An offset bayonet forcep is shown in FIG. 4, as shown offset so that the operator can see what is the object of interest and not be blocked by one's hand or fingers.

An embodiment uses forceps that have electrocautery incorporated. The purpose is to coagulate the material between the forcep tips. Often this is used to stop bleeding. Visualization is critical. The embodiment places the visualization at the tip of the instrument using the sleeve on the tube of the cannula.

The wires associated with power and viewing can be tethered to the arm of the forcep blade.

In another embodiment, the covering has an associated wireless communicator 298, also powered by the power lines 299. The wireless communicator communicates the signals that are received by the sensor 355, facilitating fewer wires to travel along the outside of the instrument

As previously noted, visualization of the tissue/object that is of interest is critical. This addition to the device allows close, on target visualization. The covering is moveable to achieve optimal field of view and clarity as well as being least intrusive to the procedure. In FIG. 4 embodiment, there can also be a rigid wire 404, tethered by clips 406 to the outside surface 402 of the tube. The wire allows the covering to move the sleeve backwards and forwards on the outer surface of the tube. In this embodiment, the holder be formed of a material that easily moves on the tube, such as an elastomeric material with which is lubricated or semi-lubricated. This can allow the sleeve to be moved on the material or on the outside of the tube from the outside of the device.

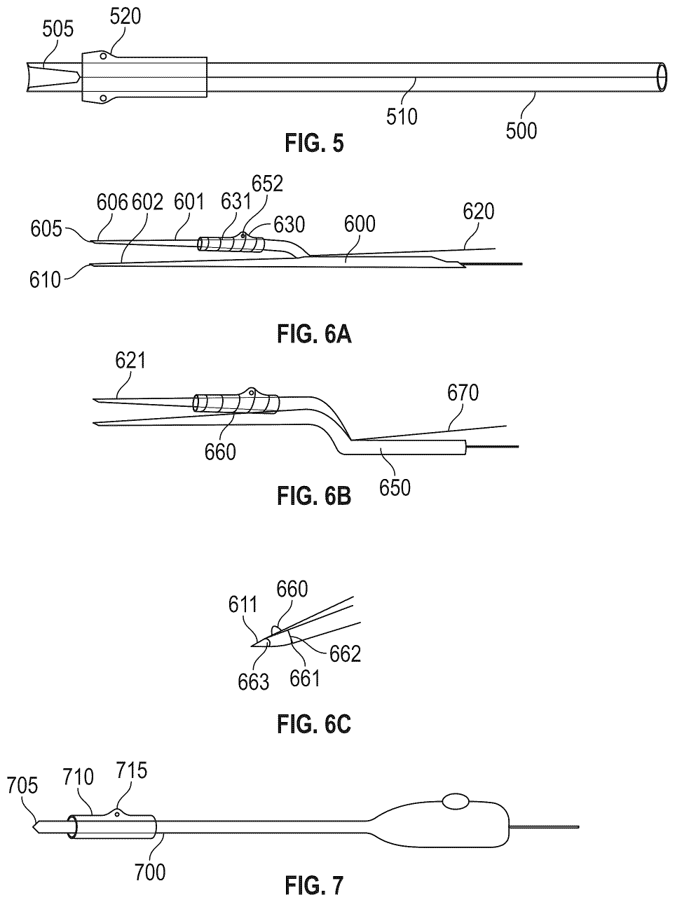

FIG. 5 shows an embodiment for delivering a surgical implant into a body such as a balloon, stent, or other expandable device. The surgical implant is delivered through a tube at 500 as in the other embodiments. The tube can be a catheter, cannula, or any surgical means of delivering a surgical implant. FIG. 5 shows the surgical implant being a surgical balloon 505. However, the surgical implant can be any other kind of surgical implant, such as a capsule, a stent, a mesh for occlusion, or a means of opening or widening a space or vessel.

The catheter often has a placement wire (trocar 510) inside the tube 500. The catheter and placement wire are separate. The sleeve 520 with sensors and light is placed on the outside of the cannula and optionally movable from outside the surgical operation. This provides a clear “window” at the catheter tip.

Visual insertion of an intracranial catheter, is used for ventriculostomy. This can be done for closed or open head trauma, and often leads to the need for relief and monitoring of intracranial pressure. The standard method of this treatment is called a ventriculostomy. A catheter is placed through a burr hole, hoping to get into the ventricular space in order to adequately manage the intracranial pressure. Using current technology, this is done “blindly”. Many believe that this leads to inaccurate placement of the catheter between 20 to 40% of the time. These procedures often are done on an emergency basis. Proper placement on the first attempt would greatly improve this relatively common procedure. Our miniaturized micro sensor is used on the ventricular catheter or at the tip on the insertion trocar. This will offer the attendant physician visual confirmation of placement on the first pass.

The previous embodiments have described how the covering for the visualization holder can be located on the cannula that houses the surgical device that is inserted for the medical operation. However, the covering can also be located directly on the actual device doing the operation. In the FIG. 2 embodiment, the clip could be directly located, for example, on the surface of the cutter 200, with the open edge of the clip facing the cutting edge 202 of the cutter.

FIGS. 6A and 6B show an embodiment where the visualization holder and covering are located directly on the operating device rather than being located on a cannula that through which the operating device is inserted. FIGS. 6A and 6B show forceps with cautery, also including a visualization. Forceps are a very common surgical/procedural tool. They are mostly to grasp items, and also can have electro cautery added, or thermal cautery added. The cautery is most often for management of hemostasis, and can also used or ablative results.

The forceps 600 have tips 605, 610, with the tip 605 including the cautery attachment 606 powered by a power cord 620. The power cord 620 is also used to provide power to the covering 630 which is placed over one of the arms of the forceps. This can be a sleeve, or a clip to leave the opening between the two arms 601, 602. The covering 630, as in other embodiments, includes LEDs 631 and camera 632 and/or.

FIG. 6B in show shows a bayonet style forceps 650, also including a visualization sleeve 660 driven by an attachable power cord 670. The sleeve 660 has the sleeve opening at the location on the forcep arm 660 where the two forcep parts come together, that is the surface of the surgical instrument that is touched to a body surface to carry out the surgical operation. FIG. 6C shows a view of the forceps showing the opening 663 between the facing sections 661, 662. The open area 663 between the two facing sections 661 662 surround and leave space for the surface that is touched to the body surface and also touched to another section of the forceps.

The accuracy of placing the cautery at precisely the area of concern is most apparent. This device will give visualization, close up, on target and also will validate the effect of the action by seeing if any post bleeding persists or even oozing that might be overlooked by more remote visualization, such as a microscope. The same is true of any ablative procedures that close inspection would better validate the effect of the treatment.

FIG. 7 shows a hand held cautery device of an embodiment. This can be electric; monopolar or bipolar, or thermal devices which are commonly used in during procedures. The device has an insertable portion 700 which can be a cautery tip 705. The visualization holder 710 is located close to the tip, with its LEDs and embedded sensors 715.

The cautery device needs to come into proximity with the desired point of the cautery. Visualization is critical to achieve the desired outcome and minimize any associated tissue/structural damage. The visualization holder is located on the shaft of the cautery instrument to allow movability and optimal visualization up close and on target for the application of the energy and for the post treatment inspection and validation.

The surgical instrument with its visualization holder can be used in various applications. An embodiment is used in Guided Ventriculostomy to reduce intracranial pressure that has becomes too high.

The current standard of care in such cases is to make a bone hole in the skull, and place a cannula into the cranial vault and try to guide it to its destination. The procedure is referred to as a “blind” insertion.

Commonly these catheter-like devices turn out to be incorrectly positioned or at times occluded and thereby not functioning properly. Replacement is therefore necessary in between 20-40% of the time.

This uses any of the previous surgical devices, or any other surgical device, with the visualization sleeve placed over the cannula.

Another embodiment places a pressure sensor in the wall of the visualization holder as one of the sensors. This can automatically sense the pressure in the location of the catheter, to allow the surgeon to determine by monitoring of that pressure if the catheter has been located into the correct high pressure location to reduce the pressure.

This system can also be used with filters.

It is known that some malignant tumors metabolize and store compounds differently than normal tissue.

This fact is used in fluorescent assisted surgery to identify the abnormal tissue and differentiate the abnormal tissue from the normal tissue. This differentiation can be particularly critical at the interface between the two tissues. This can also be especially important in tumors that have a propensity to infiltrate into normal tissue.

An accepted method of surgery is to deliver a fluorophore that is deposited in the malignant cells preferentially. A filter is used to identify the malignant cells.

In practice, the filter is placed on the operating microscope and the surgery is performed in the surgical site which is often a cavity. The core of the tumor usually fluoresces much more vividly than the margins where the infiltrative cells are located.

An embodiment describes using the visualization sensor located on the tip or on a cannula shaft of the surgical instrument to carry out the tumor visualization using a surgical direct visualization using a high acuity micro sensor. This sensor allows more careful interrogation of the tumor margins than has been possible with existing systems.

In an embodiment, an additional sleeve can be located over the visualization holder, where that additional sleeve includes a filter for optimal viewing of fluorescent signatures. Sleeves containing different filters could be interchanged to match the proper wavelength of the fluorophore.

Different kinds of agents can be used that demonstrate fluorescent properties that are not intracellular or products of tumor cellular metabolism. These agents act in additional manners to aid in delineation of tumor margins and also are used to delineate areas for blood leakage.

This embodiment shown in FIG. 8, shows a sheath covering, which covers the visualization sensor. The sheath covering has a filter at the distal end that matches to the proper wavelength to stimulate fluorescence.

This device can use multiple sheaths to optimize the visualization of the tumor margin directly with high acuity at the point of resection, without need for a microscope or optical loupes.

### Surgical Device

In another embodiment the sensor and LED illuminator is provided directly on the tip of the device. This can be located on the tip of the cautery device, scissor, forceps, automated or manual cutters, suction devices, insertion devices for hardware or biological, or biopsy devices.

The device can also be used with spine surgery applications.

The device can be used outside of medical surgery in any kind of material inspection. In this embodiment, an inspection instrument is used to carry out a function. This can be done during a quality control, trouble shooting or manufacturing defect analysis. The inspection instrument is inserted into the area of interest in a metal, plastic, composite or glass material. The instrument is covered by a visualization holder, which is sized to fit over an outside surface of the at least one inserted surface when the inspection instrument is inserted into the material, the visualization holder including at least a camera and a light emitting part, the lighting part being energized to emit light, and the camera providing an image indicative of an area of the operation lit by the lighting part. The visualization holder is held on the location on the outside surface. The holder can have a moving connection piece, which extends from the visualization holder to a location outside of the material being examined. The moving connection piece can be a cable that extends along the outside of the inserted surface, to the outside, that allows moving the connection piece along the inserted surface, back and forth. The moving connection piece can be a thumbscrew that is twisted to move the visualization holder along the surface of the inspection instrument device. The movement of the visualization holder can move the camera and light out of the path of fluids such as blood, to keep a better visualization. The movement can also change the depth of field of the camera to improve the visualization.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. For example, engineers will note that the devices and disclosures in this invention could be used to produce inspection instruments with improved visualization during product defect analysis, manufacturing trouble shooting, system performance improvement and manufacturing failure analysis for a variety of technical fields in the glass, metal, plastic and composite industries. Such inspection instruments can show areas of interest that otherwise could not be observed. The disclosures in this invention are material for any technical process or product manufacturing where improved visualization on a small scale can produce benefit not before realized. Product or manufacturing visualization systems which can change the depth of field during the inspection examination can be of significant value in identifying the area of interest, problem, defect or change required.

Thus, the present invention is not intended to be limited to the embodiments shown herein but is to be accorded the widest scope consistent with the principles and novel features disclosed herein.

What is claimed is:

1. A visualization system for a surgical instrument, comprising:
a surgical instrument which is used to carry out a function during a surgical operation, the surgical instrument being inserted into an opening in a living body during the surgical operation, the surgical instrument having at least one inserted surface that is inserted to into the body during the surgical operation to carry out the surgical function; and
a visualization holder, which is sized to fit over an outside surface of the at least one inserted surface when the surgical instrument is inserted into the body, the visualization holder including at least a camera and a light emitting part, the lighting part being energized to emit light, and the camera providing an image indicative of an area of the operation lit by the lighting part;
the visualization holder being held on a location on the outside surface relative to a location where the operation is taking place and being movable on the outside surface.

2. The system as in claim 1, wherein the visualization holder is formed of a flexible material that fits over an outer surface of the inserted surface.

3. The system as in claim 2, wherein the visualization holder is a sleeve that fits completely around the operative surface.

4. The system as in claim 2, wherein the visualization holder is a clip, which fits around only a portion of the operative surface, but which leaves an opening between two facing sections of the clip, formed to allow the clip to be clipped over the outside surface at any location along the operative surface, and leaving an open area between the two facing sections where it does not fit completely around the operative surface.

5. The system as in claim 4, wherein the surgical instrument has a surface that is touched to a body surface to carry out the surgical operation, and where the open area between the two facing sections surround and leave space for the surface that is touched to the body surface.

6. The system as in claim 1, wherein the surgical instrument is an aspiration tool.

7. The system as in claim 1, wherein the surgical instrument is a cautery device.

8. The system as in claim 1, wherein the surgical instrument is a device for implanting a surgical implement.

9. The system as in claim 1, wherein the inserted surface includes a cannula, and the surgical instrument is inside the cannula, and the visualization holder is located around an outside of the cannula.

10. The system as in claim 9, further comprising a cable running to the visualization holder, the cable being attached to the cannula.

11. The system as in claim 9, wherein the visualization holder is movable along the cannula to different locations on the cannula to adjust a depth of field obtained from the camera.

12. The system as in claim 1, wherein the visualization holder is movable along a surface of the surgical instrument to adjust a depth of field obtained from the camera.

13. The system as in claim 12, wherein further comprising a moving connection piece, which extends from the visualization holder to a location outside of the body, and enables moving the visualization holder along the surface of the surgical instrument, to change depth of field received by the camera.

14. A method of visualization during a surgical operation, comprising:
Inserting an inserted surface of a surgical instrument into a human body to carry out a function during a surgical operation, the surgical instrument being inserted into an opening in a living body during the surgical operation; and
Prior to inserting, attaching a visualization holder, which is sized to fit over an outside surface of the inserted surface when the surgical instrument is inserted into the body, over the inserted surface, where the visualization holder including at least a camera and a light emitting part, the lighting part being energized to emit light, and the camera providing an image indicative of an area of the operation lit by the lighting part;
the visualization holder being held on a location on the outside surface relative to a location where the operation is taking place while carrying out an operation; and
moving the visualization holder on the outside surface to adjust a depth of field of an image obtained from the camera.

15. The method as in claim 14, wherein the visualization holder is formed of a flexible material that fits over an outer surface of the inserted surface.

16. The method as in claim 15, wherein the visualization holder is a sleeve that fits completely around the operative surface, and the attaching comprises sliding the visualization holder over the outside surface.

17. The method as in claim 15, wherein the visualization holder is a clip, which fits around only a portion of the operative surface, but which leaves an opening between two facing sections of the clip, formed to allow the clip to be clipped over the outside surface at any location along the operative surface, and leaving an open area between the two facing sections where it does not fit completely around the operative surface, and the attaching comprises opening the clip at the open area and clipping the visualization holder over the outside surface.

18. The method as in claim 17, wherein the surgical instrument has a surface that is touched to a body surface to carry out the surgical operation, and where the open area between the two facing sections surround and leave space for the surface that is touched to the body surface.

19. The method as in claim 14, wherein the inserted surface includes a cannula, and the surgical instrument is inside the cannula, and the visualization holder is located around an outside of the cannula.

20. The method as in claim 19, further comprising a cable running to the visualization holder, the cable being attached to the cannula.

21. The method as in claim 19, wherein the visualization holder is movable along the cannula to different locations on the cannula to adjust a depth of field obtained from the camera.

22. The method as in claim 14, further comprising moving the visualization holder is movable along a surface of the instrument to adjust a depth of field obtained from the camera.

23. The method as in claim 22, further comprising a moving connection piece, which extends from the visualization holder to a location outside of the body, and the moving the visualization holder along the surface of the surgical instrument uses the moving connection piece, to change depth of field received by the camera.

24. A method of visualizing during a surgical operation, comprising:
Attaching a movable, adjustable micro sensor on a visualization holder;
Attaching the visualization holder with the micro sensor on an inserted part of an instrument participating in the surgical operation.

25. The method as in claim 24, wherein the visualization holder is a sheath that fits directly over the inserted part.

26. The method as in claim 24, wherein the visualization holder is a clip that clips directly over the inserted part.

27. A visualization device for material inspection comprising:
an inspection instrument which is used to carry out a function during a quality control, troubleshooting or manufacturing defect analysis, the inspection instrument being inserted into an area of interest in a metal, plastic, composite or glass material; and
a visualization holder, which is sized to fit over an outside surface of the inserted surface when the inspection instrument is inserted into the material, the visualization holder including at least a camera and a light emitting part, the lighting emitting part being energized to emit light, and the camera providing an image indicative of an area of the area of interest lit by the lighting part;
the visualization holder being held on a location on the outside surface.

28. The device as in claim 27, further comprising a moving connection piece, which extends from the visualization holder to a location outside of the material being examined, and the moving the visualization holder along the surface of the inspection instrument device uses the moving connection piece, to change depth of field of the device.

29. The device as in claim 27, wherein the instrument is a surgical instrument and the area of interest is a body cavity.

30. A visualization system for an instrument, comprising:
an instrument which is used to carry out a function by being inserted into an inside of a closed cavity, where that inside cannot be seen from an outside of the closed cavity;
the instrument having at least one inserted surface that is inserted into the closed cavity during operation to carry out the function; and
a visualization holder, which attaches to a surface of the at least one inserted surface when the instrument is inserted into the closed cavity, the visualization holder including at least a camera, the camera providing an image indicative of an area inside the inserted surface.

31. The system as in claim 30, wherein the visualization holder also includes a light emitting part, the lighting part being energized to emit light.

32. The system as in claim 30, wherein the instrument is a surgical instrument and the closed cavity is a body cavity.

33. The system as in claim 30, wherein the surface is an outside surface, and the visualization holder is sized to fit over the outside surface.

34. The system as in claim 30, where the visualization holder being held on a location on the outside surface relative to a location where the operation is taking place and being movable on the outside surface.
